(19) United States
(12) Patent Application Publication
(10) Pub. No.: US 2022/0361876 A1
(43) Pub. Date: Nov. 17, 2022

(54) Bio-Zipper Surgical Closure Device

(71) Applicant: THE REGENTS OF THE UNIVERSITY OF CALIFORNIA, Oakland, CA (US)

(72) Inventors: Renea Sturm, Los Angeles, CA (US); George Aninwene, Oakland, CA (US); Peyton Tebon, Oakland, CA (US); Mohammad Ali Darabi, Oakland, CA (US); Alireza Khademhosseini, Oakland, CA (US)

(21) Appl. No.: 17/769,686

(22) PCT Filed: Oct. 15, 2020
(86) PCT No.: PCT/US20/55766

§ 371 Date: Apr. 15, 2022

Related U.S. Application Data
(60) Provisional application No. 62/915,361, filed on Oct. 15, 2019.

(51) Int. Cl. A61B 17/08

(52) U.S. Cl. CPC A61B 17/085; A61B 2017/00004

(57) ABSTRACT

The present invention relates to bio-zipper surgical closure devices configured to provide tension-free support of an incision throughout the healing process. Further, the present invention provides a surgical closure device to be used for urethral tubular closure during a urethroplasty. The present invention relates to methods of using the surgical closure device for the closure of various wounds such as urethral tubular closure during a urethroplasty.

### CROSS-REFERENCE TO RELATED APPLICATIONS

This application claims priority to U.S. Provisional Patent Application No. 62/915,361, filed Oct. 15, 2019, the contents of which are incorporated by reference herein in its entirety.

### BACKGROUND OF THE INVENTION

In adults, a condition in which a urethroplasty is the gold standard intervention is urethral stricture disease with narrowing of the urethra as may occur secondary to traumatic injury. Urethral stricture disease is estimated to account for >200,000 patient visits per year, with a total of 13,700 adult men undergoing urethroplasty nationally between 2000-2010 (Blaschko, S. D. et al., 2015, Urol., 85(5):1190-94). Regardless of indication for urethroplasty, short- and long-term complications include fistula formation (abnormal secondary urethral opening from tube to shaft skin), dehiscence (break-down of the urethral tube), diverticulum or stricture formation (abnormal enlargement or narrowing respectively of a portion of the tube). Each of these defects is increased in the setting of abnormal wound healing which may be observed in particular in the setting of localized tension or inadequate vascularization leading to fibrosis within the region of urethral repair.

Depending upon the severity of the defect, tissue utilized in urethral creation includes remnant urethral tissue, foreskin and/or tissue from the inside of the check or lip (buccal). This is placed into the defect as a graft or flap with a urethroplasty completed after vascularization occurs from the corporal base in 6 to 12 months. During this second stage, current standard of care ventral urethroplasty closure in hypospadias is completed in the ventral midline in 2 to 3 layers using small absorbable sutures such as a 6-0 or 7-0 Vicryl (polyglactic acid), PDS (polydioxanone), or Monocryl (poliglecaprone 25). Although controversial due to potential risks of inflammation and/or infection associated with catheterization, a urethral catheter or stent typically remains in place for 1 to 3 weeks postoperatively in complex or proximal cases to prevent local urine leak and local tension on the urethral tissue. However, it is notable that the small lumen catheters (6 or 8F) suitable for use in children may develop occlusion or dislodgement and, like all urethral catheters, can increase discomfort as well as increase the risk of urinary tract infection. Indeed, a recent series demonstrated that 36% of urethral catheters placed during hypospadias surgery required intervention or led to an ER visit due to stent related complications (Lee, L. C. et al., 2018, JPU, 14:423e1-5) making this the primary driver of visits to the ER in the perioperative period following a urethroplasty.

Thus, there is a need in the art for a closure device and a method to improve urethroplasty outcomes that optimizes urethral healing to decrease complications, improve wound healing, facilitate urination without a urethral catheter, provide a stable sealant mechanism that would minimize urine leak without catheter and prevent localized tension on the wound closure. The present invention meets this need.

### SUMMARY OF THE INVENTION

In one aspect the present invention relates to a bio-zipper surgical closure device comprising: a flexible base; and a plurality of microstructures, each comprising a proximal end, a distal end, a body and a tip protruding from the base. In one embodiment, the microstructures are selected from the group consisting of microneedles, microblades, microanchors, microfishscale, micropillars, microhairs and combinations thereof. In one embodiment, the microstructures each comprise a tip diameter ranging from about 10 nm to about 1 μm. In one embodiment, the microstructures each comprise a length ranging from about 1 μm to about 2 mm. In one embodiment, the base is biodegradable. In one embodiment, the plurality of microstructures are biodegradable. In one embodiment, the plurality of bio-zipper devices are linked together via a flexible backbone. In one embodiment, the plurality of bio-zipper devices are placed adjacent together leaving a space between each bio-zipper ranging between about 0 to about 1cm. In one embodiment, the plurality of bio-zipper devices are linked together via a closure member, wherein the closure member allows the at least two adjacent bio-zippers to be drawn closer together. In one embodiment, the closure member is selected from the group consisting of a suture, a pull tab and combinations thereof

In another aspect, the present invention relates to a biotape surgical closure device comprising: a right panel; a left panel; and a closure member, wherein the closure member is configured to allow the right panel and the left panel to be drawn close together. In one embodiment, the closure member is selected from the group consisting of a suture, a pull tab and combinations thereof. In one embodiment, the right panel and the left panel are made from adhesive material. In one embodiment, the right panel and the left panel comprise Poly (glycerol sebacate) (PGS).

In another aspect, the present invention relates to a method for wound closure comprising: providing a bio-zipper surgical closure device, wherein the bio-zipper surgical closure device comprises a flexible base and a plurality of microstructures, wherein each microstructure comprises a proximal end, a distal end, a body and a tip protruding from the base; aligning and abutting edges of a tissue wound to be joined; securing at least one microstructure to the tissue on one side of the wound; stretching the bio-zipper surgical closure device across the wound so as to secure at least one microstructure to the tissue on the opposing side of the wound. In one embodiment, the microstructures are selected from the group consisting of microneedles, microblades, microanchors, microfishscale, micropillars, microhairs and combinations thereof. In one embodiment, the microstructures each comprise a tip diameter ranging from about 10 nm to about 1 μm. In one embodiment, the microstructures each comprise a length ranging from about 1 μm to about 2 mm. In one embodiment, the base is biodegradable. In one embodiment, the plurality of microstructures are biodegradable.

In another aspect, the present invention relates to a method for wound closure comprising: providing a bio-zipper surgical closure device comprising a plurality of bio-zippers attached together via a backbone, wherein the bio-zipper device comprises a flexible base and a plurality of microstructures protruding from the base and wherein the plurality of bio-zippers can be drawn together via a closure member; aligning and abutting edges of a tissue wound to be joined; securing at least one microstructure from the at least one bio-zipper to the tissue on one side of the wound; stretching the bio-zipper surgical closure device across the wound so as to secure at least one microstructure from at least one bio-zipper to the tissue on the opposing side of the wound; using closure members to close the tissue wound by pulling the abutting edges of the wound closer to each other. In one embodiment, the microstructures are selected from the group consisting of microneedles, microblades, microanchors, microfishscale, micropillars, microhairs and combinations thereof. In one embodiment, the microstructures each comprise a tip diameter ranging from about 10 nm to about 1 μm. In one embodiment, the microstructures each comprise a length ranging from about 1 μm to about 2 mm. In one embodiment, the base is biodegradable. In one embodiment, the plurality of microstructures are biodegradable.

In another aspect, the present invention relates to a method for wound closure comprising: providing a biotape surgical closure device comprising a right panel, a left panel and a closure member, wherein the closure member is configured to allow the right panel and the left panel to be drawn close together; aligning and abutting edges of a tissue wound to be joined; securing the right panel to the tissue on one side of the wound and securing the left panel to the tissue on the opposing side of the wound; using closure members to close the tissue wound by pulling the abutting edges of the wound closer to each other.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A through FIG. 1B depict a perspective view of an exemplary bio-zipper surgical closure device. FIG. 1A depicts a perspective view of an exemplary bio-zipper surgical closure device placed on 3d printed model of a urethra. FIG. 1B depicts a perspective view of an exemplary bio-zipper surgical closure device.

FIG. 2 depicts a perspective view of multiple exemplary bio-zipper surgical closure device of the present invention.

FIG. 3 depicts a perspective view of an exemplary biotape surgical closure device of the present invention.

FIG. 4 depicts a perspective view of another exemplary biotape surgical closure device of the present invention.

FIG. 5 depicts a perspective view of another exemplary biotape surgical closure device of the present invention.

FIG. 6 is a flowchart depicting an exemplary method of wound closure using an exemplary bio-zipper surgical closure device of the present invention.

FIG. 7 is a flowchart depicting an exemplary method of wound closure using an exemplary bio-zipper surgical closure device of the present invention.

FIG. 8 is a flowchart depicting an exemplary method of wound closure using an exemplary biotape surgical closure device of the present invention.

FIG. 9 comprising FIG. 9A and FIG. 9B depicts current standard of care in lower urinary tract reconstruction. FIG. 9A depicts Hautmann et al. neobladder (Hautmann, R. E. et al., 2015, Urology, 85:233-238). FIG. 9B depicts augmentation cystoplasty prior to reservoir completion.

FIG. 10 comprising FIG. 10A through FIG. 10C depicts synthesis of Poly(glycerol sebacate) (PGS) and the mechanical properties of PGS. FIG. 10A depicts that PGS is synthesized by the polycondensation of glycerol and sebacic acid. FIG. 10B depicts Stress—strain curves for PGS as a function of curing time. FIG. 10C depicts Young's modulus (YM) for PGS as a function of curing time.

### DETAILED DESCRIPTION

It is to be understood that the figures and descriptions of the present invention have been simplified to illustrate elements that are relevant for a clear understanding of the present invention, while eliminating, for the purpose of clarity, many other elements found in the field of surgical devices, including those indicated for the treatment of peripheral nerve anastomosis. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of±20%, ±10%,±5%,±1%, or±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Bio-Zipper Surgical Closure Device

The present invention relates in part to a bio-zipper surgical closure device, an implantable wound closure device for use in a subject. In one embodiment, the bio-zipper surgical closure device provides tension-free support of an incision throughout the healing process. In one embodiment, the present invention provides a bio-zipper surgical closure device suitable for urethral tubular closure during a urethroplasty. The bio-zipper device is designed to facilitate epithelial inversion, minimize urine leak, alleviate tension along the full extent of a ventral urethral closure site, and prevent localized laminar flow effects.

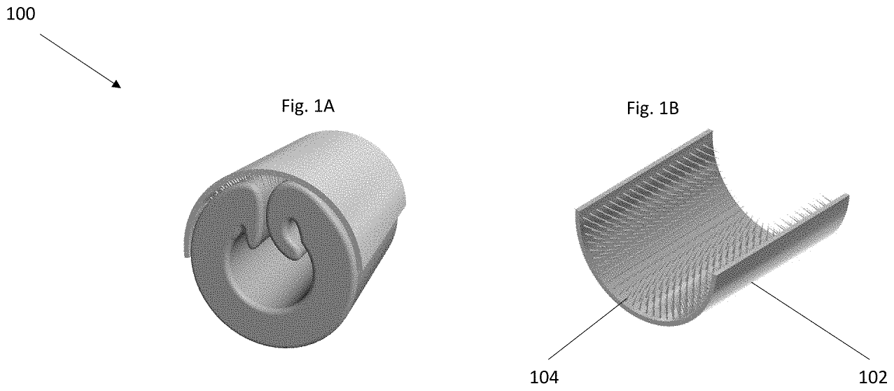

Referring to FIG. 1A and FIG. 1B, an exemplary bio-zipper device 100 of the present invention is shown. Bio-zipper 100 comprises a base 102 and a plurality of microstructures 104 protruding from base 102.

Base 102 can be made of a stretchable and breathable material. Alternatively, Base 102 can be made of any suitable material. In some embodiments, for example, base 102 can be made of a material that is transparent, or substantially transparent, thus allowing for non-invasive monitoring of wound healing. In other embodiments, base 102 can be made of a material that is not transparent. In one embodiment, base 102 may be made from natural, synthetic, and/or artificial materials; and in some particular embodiments, they comprise a polymeric substance (e.g., a silicone, a polyurethane, or a polyethylene). Base 102 may be comprised of materials that are nontoxic, biodegradable, bioresorbable, or biocompatible. In some embodiments, base 102 comprise inert materials, and in other embodiments, base 102 comprises activated materials, (e.g., activated carbon cloth to remove microbes, as disclosed in WO2013028966A2, incorporated herein in its entirety). In some embodiments, base 102 comprise a material singularly, or in combination, selected from the group consisting of medical tape, white cloth tape, surgical tape, tan cloth medical tape, silk surgical tape, clear tape, hypoallergenic tape, silicone, elastic silicone, polyurethane, elastic polyurethane, polyethylene, elastic polyethylene, rubber, latex, Gore-Tex, plastic and plastic components, polymers, biopolymers, and natural materials.

Alternatively, the flexibility and/or stretchability of base 102 may vary across, or along, bio-zipper 100. Further, in some embodiments, base 102 can comprise elastic properties, wherein the elasticity may optionally be similar throughout base 102. Alternatively, the elasticity may be varied along or across base 102.

The degree of flexibility of base 102 is determined by the material of construction, the shape and dimensions of the device, the type and properties of the approximated tissue, and the area of the body into which bio-zipper 100 is placed. For example, a tightly curved or mobile part of the body, e.g. a joint, may require a more flexible base, as would a tendon or nerve repair due to the amount of bending bio-zipper 100 needs for the attachment. Also, depending on the type of material used, the thickness of base 102 as well as its width and length may determine the flexibility of the device. Thickness of base 102 can be in a range between about 10 μm to 1 cm. Base 102 may be pre-fabricated into different shapes. In one embodiment, base 102 has sharp corners. In one embodiment, base 102 has round corners. The shape and dimensions of base 102 can be modified to change the flexibility of bio-zipper 100.

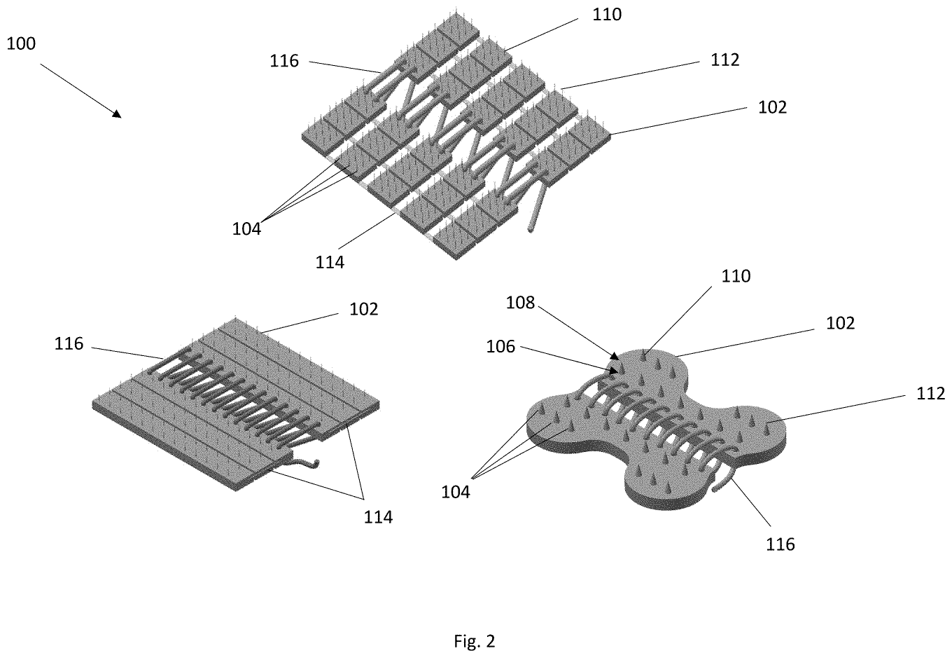

Referring now to FIG. 2, microstructures 104 each comprise a proximal end 106, a distal end 108, a body 110 and a tip 112. Microstructure 104 can be either straight or curved. In some embodiments, body 110 connects proximal end 106 to distal end 108 without curvature along its length. In one embodiment, body 110 is curved along its length between proximal end 106 to distal end 108.

Shapes of microstructures 104 may be varied depending, e.g. on the area of the body involved and the type of tissue requiring closure or re-approximation. Microstructures 104 may be canted or erect. In one embodiment, the general structure of microstructures 104 is of a rose thorn shape. In one embodiment, microstructures 104 are selected from the group consisting of microneedles, microblades, microanchors, microfishscale, micropillars, microhairs, and combinations thereof. Microstructures 104 can have a sharp tip 112 enabling it to penetrate into tissue, or can have a blunt tip 112 that enables it to merely grasp tissue without actual penetration. In one embodiment, microstructures 104 are designed to penetrate tissue to specific depths.

Microstructures 104 can have a circular cross-section or non-circular cross-section at proximal end 106. The cross-sectional dimensions typically are between about 10 nm and 1 mm, preferably between about 1 micron and 200 microns, and more preferably between about 10 and 100 μm.

The bio-zipper 100 of the present invention may comprise microstructures 104 of any desired size, dimension, and geometry. Additionally, microstructures 104 may optionally comprise surfaces which are substantially smooth, or which comprise uneven surfaces, e.g., a microstructure comprising sides which are wavy, or which comprise protrusions, indentations, or depressions. For example, body 110 can have concave surfaces, convex surfaces, or a combination of concave and convex surfaces. In one embodiment, body 110 comprises at least one concave surface. In one embodiment, body 110 comprises at least one convex surface. In one embodiment, body 110 comprises at least one concave surface and at least one convex surface. Tip 112 is located at distal end 108. In one embodiment, tip 112 can be selected from a group consisting of: a cube, a rectangle, a sphere, a cone, a pyramid, a cylinder, a tube, a ring, a tetrahedron, a hexagon, an octagon, or any irregular shapes. In one embodiment, the dimension (e.g., a diameter) of tip 112 may be within a range of about 10 nm to 1 μm. The density, distribution, length, and orientation of microstructures 104 on base 102 may be modified depending on the type of wound closure. Microstructures 104 may be bent or curve gradually, with distal end 108 directed at an optimal angle relative to base 102 to aid device penetration and stability within the tissue, and to reduce tissue irritation after installation. Microstructures 104 may be canted in one direction, such as toward the center of bio-zipper 100. Microstructures 104 may also be variously oriented, such as toward center and erect, or toward center and away from center. It is within the scope of this invention to have microstructures 104 extending in any relative direction or orientation on base 102.

In one embodiment, bio-zipper 100 of the present invention comprises microstructures 104 at an angle relative to base 102. Microstructures 104 may be positioned at any suitable angle. In one embodiment, microstructures 104 are affixed at an angle relative to base 102, wherein the angle is approximately 15, 30, 45, 60, 75, or 90 degrees, including all integers (e.g., 16°, 17°, 18°, etc.) and ranges (e.g., 15°-90°, 30°-90°, 45°-70°, etc.) in between of the angles set forth. In one embodiment, bio-zipper 100 of the present invention also include microstructures 104 with an angle relative to base 102, that is variable depending on its position in any microstructure array. In certain embodiments, the angle of one or more microstructures 104 is approximately constant along the entire length of the microstructure 104, and in other embodiments, the angle of the microstructure 104 varies along the length of the microstructure 104.

Microstructures 104 may be angled in any direction. In some embodiments, all microstructures 104 in a particular array are angled in the same direction, or in approximately the same direction; while in other embodiments they are not.

In one embodiment, microstructures 104 of various lengths emanate from a single base 102. For example, in one embodiment, microstructures 104 are progressively shorter the closer they are to the center of bio-zipper 100. In one embodiment, microstructures 104 may also become progressively shorter the farther they are from the center of bio-zipper 100. In one embodiments, the length of an individual microstructure 104 may be within a range of about 1 μm to 2 mm. It may be desirable, in certain embodiments, to adjust the length of a microneedle according to the application/use and/or a payload delivered by bio-zipper 100.

The density of microstructures 104 may be predetermined and may vary depending upon the size of bio-zipper 100 and the wound to be closed, much as bandages vary in size and the location on the body where they are to be applied. In one embodiment, the density may be about or greater than about 100,000/cm², about 10,000/cm², about 5,000/cm², about 1,000/cm², about 500/cm², about 100/cm², about 50/cm², about 10/cm², or even about 1/cm². The pitch between adjacent microneedles may be from about 10 μm to more than 1 cm, wherein pitch is defined as the distance between microstructures 104, center point to center point.

Materials used for a microstructure 104 or a portion thereof may be selected and adapted for a particular use or design. Microstructures 104 can comprise a therapeutic agent. For instance, a therapeutic agent can be used in its crystallized or lyophilized state. In one embodiment, microstructures 104 can comprise a degradable polymer. Without wishing to be bound by any particular theory, the degradable portion of microstructures 104 and the degradation rate may dictate the mechanism and efficiency of delivery of a therapeutic agent or other functions of bio-zipper 100. For instance, microstructure 104 can include or introduce a therapeutic agent so that the therapeutic agent is released after the degradation of microstructure 104. In one embodiment, base 102 comprises a degradable material. In certain embodiments, base 102 degrades so that microstructure 104 is released from bio-zipper 100 and may remain lodged in the internal tissue after interaction and/or implantation. In certain embodiments, microstructure 104 lodged in the internal tissue may gradually degrade. In one embodiment, tip 112 comprises a degradable material. In one embodiment, tip 112 of a microstructure 104 degrades so that only tip 112 of the microstructure 104 breaks off. In one embodiment, microstructures 104 may be coated with a therapeutic agent. In one embodiment, base 102 may be coated with a therapeutic agent.

Suitable degradable polymers, and derivatives or combinations thereof, as discussed above can be selected and adapted to have a desired degradation rate. Alternatively, a degradation rate may be fine-tuned by associating or mixing other materials as previously described (e.g., non-degradable materials) with one or more of degradable polymers.

Bio-zipper 100 may comprise any material or mixture of materials. In one embodiment, bio-zipper 100 can comprise one or more biocompatible materials. Exemplary materials include, but are not limited to, metals (e.g., gold, silver, platinum, steel or other alloys); metal-coated materials; metal oxides; plastics; ceramics; silicon; glasses; mica; graphite; hydrogels; and polymers such as non-degradable or biodegradable polymers; and combinations thereof. Bio-zipper 100 may comprise one or more materials. In general, materials can be utilized in any form (e.g., lyophilized or crystallized) and/or for different purposes (e.g., therapeutics, diagnostics, etc.)

In some embodiments, bio-zipper 100 can comprise a magnetic material. For examples, a magnetic material can be utilized for positioning bio-zipper 100 in a target site or orientation, to trigger delivery of a therapeutic agent, or to affect interaction of the microstructure 104 to an internal tissue or a vessel wall.

In some embodiments, bio-zipper 100 can comprise deformable materials (e.g., polymers). As an example, bio-zipper 100 can comprise a deformable rubber so that the device swells enabling interaction of microstructure 104 protruding from base 102 to a tissue. In another example, a deformable bio-zipper 100 may be able to change size depending on pressure so that it can pass through lumens with diameters smaller than that of the device.

In some embodiments, bio-zipper 100 can comprise adhesive materials (e.g., adhesive polymers). As examples, bioadhesives such as chitosan and carbopol can be used. An adhesive material may be used to bring bio-zipper 100 close to an internal tissue or a vessel wall facilitating the interaction of microstructures 104. Adhesiveness of the bio-zipper 100 can aid in fixing/implanting at a target site for a prolonged period of time. For example, in treating an area of disease tissue, an adhesive device may act as a depot formulation for drugs used to treat chronic conditions.

In some embodiments, bio-zipper 100 can comprise one or more polymers. For example, a portion of bio-zipper 100 (e.g., microstructures 104) and/or a coating can comprise one or more polymers. Various polymers and methods known in the art can be used. Polymers may be natural polymers or unnatural (e.g. synthetic) polymers. In some embodiments, polymers can be linear or branched polymers. In some embodiments, polymers can be dendrimers. Polymers may be homopolymers or copolymers comprising two or more monomers. In terms of sequence, copolymers may be block copolymers, graft copolymers, random copolymers, blends, mixtures, and/or adducts of any of the foregoing and other polymers.

A polymer used in accordance with the present application can have a wide range of molecular weights. In some embodiments, the molecular weight of a polymer is greater than about 5 kDa. In some embodiments, the molecular weight of a polymer is greater than about 10 kDa. In some embodiments, the molecular weight of a polymer is greater than 50 kDa. In some embodiments, the molecular weight of a polymer is within a range of about 5 kDa to about 100 kDa.

In some embodiments, polymers may be synthetic polymers, including, but not limited to, polyethylenes, polycarbonates (e.g. poly(1,3-dioxan-2-one)), polyanhydrides (e.g. poly(sebacic anhydride)), polyhydroxyacids (e.g. poly((3-hydroxyalkanoate)), polypropylfumarates, polycaprolactones, polyamides (e.g. polycaprolactam), polyacetals, polyethers, polyesters (e.g. polylactide, polyglycolide), poly(orthoesters), polycyanoacrylates, polyvinyl alcohols, polyurethanes, polyphosphazenes, polyacrylates, polymethacrylates, polyureas, polystyrenes, and polyamines and copolymers thereof. In some embodiments, polymers include polymers which have been approved for use in humans by the U.S. Food and Drug Administration (FDA) under 21 C.F.R. § 177.2600, including, but not limited to, polyesters (e.g. polylactic acid, poly(lactic-co-glycolic acid), polycaprolactone, polyvalerolactone, poly(1,3-dioxan-2-one)); polyanhydrides (e.g. poly(sebacic anhydride)); polyethers (e.g., polyethylene glycol); polyurethanes; polymethacrylates; polyacrylates; polycyanoacrylates; copolymers of PEG and poly(ethylene oxide) (PEO).

In some embodiments, polymers used herein can be a degradable polymer. Such a degradable polymer can be hydrolytically degradable, biodegradable, thermally degradable, and/or photolytically degradable polyelectrolytes. For example, degradation of a bio-zipper 100 comprising a degradable polymer can be induced by the ingestion of a solution targeted to specifically degrade bio-zipper 100 or a portion of the device (e.g., at least one microstructure 104).

Degradable polymers known in the art include, for example, certain polyesters, polyanhydrides, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Another exemplary degradable polymer is poly (beta-amino esters), which may be suitable for use in accordance with the present application.

Bio-zipper 100 may be molded, stamped, machined, woven, bent, welded or otherwise fabricated to create the desired features and functional properties.

Referring now to FIG. 2, another exemplary bio-zipper surgical wound closure device is shown.

In one embodiment, a plurality of bio-zippers 100 can be attached together via a flexible backbone 114. Backbone 114 can attach to the plurality of bio-zippers 100 by any means, including but not limited to adhesives, snap fits, etc. Backbone 114 can be made of any suitable material. In some embodiments, for example, Backbone 114 can be made from natural, synthetic, and/or artificial materials; and in some particular embodiments, backbone 114 can comprise a polymeric substance (e.g., a silicone, a polyurethane, or a polyethylene). Backbone 114 may comprise materials that are nontoxic, biodegradable, bioresorbable, or biocompatible.

In one embodiment, a plurality of bio-zippers 100 are placed adjacent together leaving a space between each bio-zipper 100 ranging between about 0 to 1 cm.

This space allows the bio-zippers 100 to move flexibly and bend based on the location of the application site.

In one embodiment, at least two bio-zippers 100 can be attached together via a closure member 116. Closure member 116 allows the at least two bio-zippers 100 to be drawn closer together using sutures, pull tabs, or any other mechanism known to the skilled artisan. The action of drawing the plurality of bio-zipper together causes the edges of the tissue opening to be brought toward each other and allows certain embodiments to be effectively applied to tissue openings of varying sizes.

### Biotape Closure Device

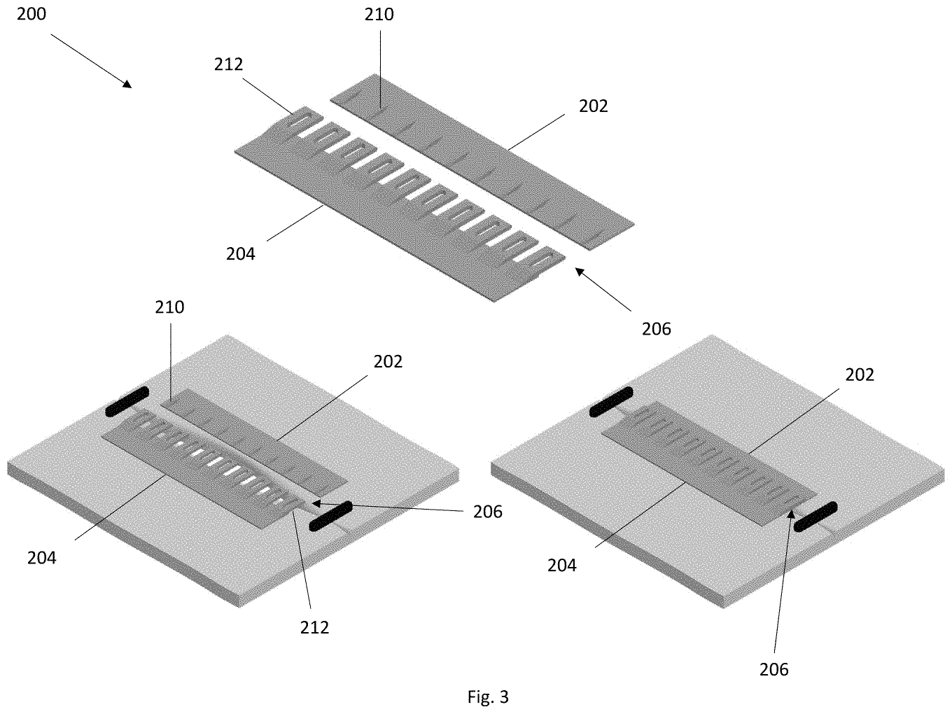

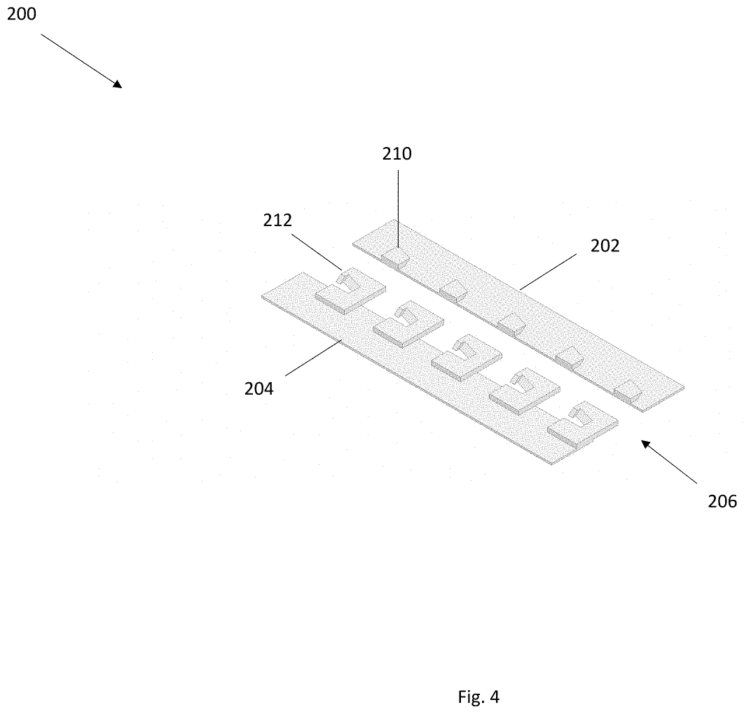

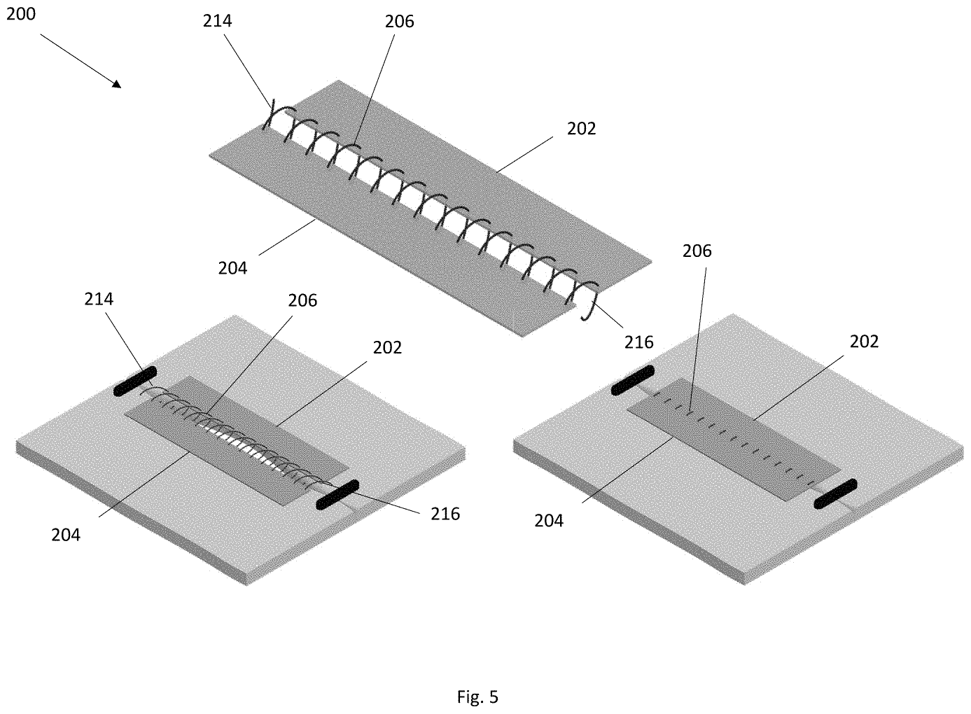

The present invention relates in part to a biotape surgical closure device, an implantable wound closure device for use in a subject. In one embodiment, the biotape surgical closure device provides tension-free support of an incision throughout the healing process. In one embodiment, the present invention provides a biotape surgical closure device suitable for urethral tubular closure during a urethroplasty. In one embodiment, the biotape device is designed to facilitate epithelial inversion, minimize urine leak, alleviate tension along the full extent of a ventral urethral closure site, and prevent localized laminar flow effects. In one embodiment, the biotape surgical closure device provides a water-tight surgical closure. Referring now to FIG. 3, FIG. 4 and FIG. 5, an exemplary biotape device 200 of the present invention is shown. Biotape 200 comprises a right panel 202, a left panel 204 and a closure member 206.

Right panel 202 comprises a lower surface and an upper surface. Similarly, left panel 204 comprises a lower surface and an upper surface. Right panel 202 and left panel 204 can be made of a stretchable and breathable material. Alternatively, right panel 202 and left panel 204 can be made of any suitable material. In some embodiments, for example, right panel 202 and left panel 204 can be made of a material that is transparent, or substantially transparent, thus allowing for non-invasive monitoring of wound healing. In other embodiments, right panel 202 and left panel 204 can be made of a material that is not transparent. In one embodiment, right panel 202 and left panel 204 may be made from natural, synthetic, and/or artificial materials; and in some particular embodiments, they comprise a polymeric substance (e.g., a silicone, a polyurethane, or a polyethylene).

Right panel 202 and left panel 204 may comprise any material or mixture of materials. In one embodiment, Right panel 202 and left panel 204 can comprise one or more biocompatible materials. Exemplary materials include, but are not limited to, metals (e.g., gold, silver, platinum, steel or other alloys); metal-coated materials; metal oxides; plastics; ceramics; silicon; glasses; mica; graphite; hydrogels; and polymers such as non-degradable or biodegradable polymers; and combinations thereof Right panel 202 and left panel 204 may comprise one or more materials. In general, materials can be utilized in any form (e.g., lyophilized or crystallized) and/or for different purposes (e.g., therapeutics, diagnostics, etc.)

In some embodiments, right panel 202 and left panel 204 can comprise a magnetic material. For examples, a magnetic material can be utilized for positioning the panels in a target site or orientation, to trigger delivery of a therapeutic agent to an internal tissue or a vessel wall.

In some embodiments, right panel 202 and left panel 204 can comprise deformable materials (e.g., polymers). As an example, right panel 202 and left panel 204 can comprise a deformable rubber so that a volume of biotape 200 can respond to external pressure. In another example, a deformable right panel 202 and left panel 204 may be able to change size depending on pressure so that it can pass through lumens with diameters smaller than that of the device.

Right panel 202 and left panel 204 may comprise materials that are nontoxic, biodegradable, bioresorbable, or biocompatible. In some embodiments, right panel 202 and left panel 204 may comprise inert materials, and in other embodiments, right panel 202 and left panel 204 may comprise activated materials, (e.g., activated carbon cloth to remove microbes, as disclosed in WO2013028966A2, incorporated herein in its entirety). In some embodiments, right panel 202 and left panel 204 may comprise a material singularly, or in combination, selected from the group consisting of medical tape, white cloth tape, surgical tape, tan cloth medical tape, silk surgical tape, clear tape, hypoallergenic tape, silicone, elastic silicone, polyurethane, elastic polyurethane, polyethylene, elastic polyethylene, rubber, latex, Gore-Tex, plastic and plastic components, polymers, biopolymers, and natural materials.

In some embodiments, right panel 202 and left panel 204 can comprise one or more polymers. For example, a portion of right panel 202 and left panel 204 and/or a coating can comprise one or more polymers. Various polymers and methods known in the art can be used. Polymers may be natural polymers or unnatural (e.g. synthetic) polymers. In some embodiments, polymers can be linear or branched polymers. In some embodiments, polymers can be dendrimers. Polymers may be homopolymers or copolymers comprising two or more monomers. In terms of sequence, copolymers may be block copolymers, graft copolymers, random copolymers, blends, mixtures, and/or adducts of any of the foregoing and other polymers.

A polymer used in accordance with the present application can have a wide range of molecular weights. In some embodiments, the molecular weight of a polymer is greater than about 5 kDa. In some embodiments, the molecular weight of a polymer is greater than about 10 kDa. In some embodiments, the molecular weight of a polymer is greater than 50 kDa. In some embodiments, the molecular weight of a polymer is within a range of about 5 kDa to about 100 kDa.

In some embodiments, polymers may be synthetic polymers, including, but not limited to, polyethylenes, polycarbonates (e.g. poly(1,3-dioxan-2-one)), polyanhydrides (e.g. poly(sebacic anhydride)), polyhydroxyacids (e.g. poly((3-hydroxyalkanoate)), polypropylfumarates, polycaprolactones, polyamides (e.g. polycaprolactam), polyacetals, polyethers, polyesters (e.g. polylactide, polyglycolide), poly(orthoesters), polycyanoacrylates, polyvinyl alcohols, polyurethanes, polyphosphazenes, polyacrylates, polymethacrylates, polyureas, polystyrenes, and polyamines and copolymers thereof. In some embodiments, polymers include polymers which have been approved for use in humans by the U.S. Food and Drug Administration (FDA) under 21 C.F.R. § 177.2600, including, but not limited to, polyesters (e.g. polylactic acid, poly(lactic-co-glycolic acid), polycaprolactone, polyvalerolactone, poly(1,3-dioxan-2-one)); polyanhydrides (e.g. poly(sebacic anhydride)); polyethers (e.g., polyethylene glycol); polyurethanes; polymethacrylates; polyacrylates; polycyanoacrylates; copolymers of PEG and poly(ethylene oxide) (PEO). In some embodiments, polymers used herein can be a degradable polymer.

Such a degradable polymer can be hydrolytically degradable, biodegradable, thermally degradable, and/or photolytically degradable polyelectrolytes. For example, degradation of right panel 202 and left panel 204 comprising a degradable polymer can be induced by the ingestion of a solution targeted to specifically degrade right panel 202 and left panel 204 or a portion of the panels.

Degradable polymers known in the art include, for example, certain polyesters, polyanhydrides, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Another exemplary degradable polymer is poly (beta-amino esters), which may be suitable for use in accordance with the present application.

In one embodiment, the flexibility and/or stretchability of right panel 202 and left panel 204 may vary across, or along, biotape 200. Further, in some embodiments, right panel 202 and left panel 204 can comprise elastic properties, wherein the elasticity may optionally be similar throughout right panel 202 and left panel 204. Alternatively, the elasticity may be varied along or across right panel 202 and left panel 204.

The degree of flexibility of right panel 202 and left panel 204 is determined by the material of construction, the shape and dimensions of the device, the type and properties of the approximated tissue, and the area of the body into which biotape 200 is placed. For example, a tightly curved or mobile part of the body, e.g. a joint, may require a more flexible base, as would a tendon or nerve repair due to the amount of bending biotape 200 needs for the attachment. Also, depending on the type of material used, the thickness of right panel 202 and left panel 204 as well as its width and length may determine the flexibility of the device. Thickness of right panel 202 and left panel 204 can be in a range between about 10 μm to 1 cm. Right panel 202 and left panel 204 may be pre-fabricated into different shapes. In one embodiment, right panel 202 and left panel 204 have sharp corners. In one embodiment, right panel 202 and left panel 204 have round corners. The shape and dimensions of right panel 202 and left panel 204 can be modified to change the flexibility of biotape 200.

In one embodiment, right panel 202 and left panel 204 may comprise Poly (glycerol sebacate) (PGS). PGS is a well-established biomaterial designed to mimic the mechanical behavior of extracellular matrix components collagen and elastin. PGS is synthesized by the polycondensation of glycerol and sebacic acid which creates a viscous prepolymer. In one embodiment, PGS may be crosslinked by increasing the temperature past the prepolymer melting point. In one embodiment, the PGS synthesis process can be leveraged to tune the mechanical properties of the final elastomer. In one embodiment, the mechanical properties of PGS can be tuned by altering the ratio of glycerol and sebacic acid during the initial synthesis step. In one embodiment, the mechanical properties of PGS can be tuned by altering the temperature in the secondary curing step. In one embodiment, the mechanical properties of PGS can be tuned by modifying crosslinking time in the secondary curing step.

In one embodiment, right panel 202 and left panel 204 may comprise an adhesive surface on their respective lower surfaces. In one embodiment, bottom surfaces of right panel 202 and left panel 204 may be covered with a pressure-responsive adhesive, where the adhesive is initially covered with a protective layer which may be peeled away immediately prior to use. In one embodiment, biotape 200 may further comprise pull-away tabs or other similar structures to hold right panel 202 and left panel 204 together at a pre-determined spaced apart distance after the protective layer has been removed but prior to adhering the panels to tissue surface.

In one embodiment, right panel 202 and left panel 204 may be made from a material with adhesive properties. In one embodiment, right panel 202 and left panel 204 having adhesive properties minimizes risk of delamination and improves mechanical stability of the device when in place.

Closure member 206 allows right panel 202 and left panel 204 to be drawn closer together using sutures, pull tabs, or any other mechanism known to the skilled artisan. The action of drawing right panel 202 and left panel 204 together causes the edges of the tissue opening to be brought toward each other and allows certain embodiments to be effectively applied to tissue openings of varying sizes.

Referring now to FIG. 3 and FIG. 4, an exemplary biotape surgical closure device 200 of the present invention is shown. In one embodiment, closure member 206 may comprise a right member 210 and a left member 212. Right member 210 is secured to an upper surface of right panel 202 and left member 212 is secured to an upper surface of left panel 204. In one embodiment, right member 210 and left member 212 are configured to couple together through a variety of coupling interfaces and bring the edges of the tissue opening toward each other. In one embodiment, the coupling interface is a snap fit mechanism (FIG. 3 and FIG. 4). Other locking interfaces, mechanisms or structures may include but are not limited to resealable adhesive layers, slide locks, locking pins and the like.

Referring now to FIG. 5, another exemplary biotape surgical closure device 200 of the present invention is shown. In one embodiment, closure member 206 may comprise a continuous strap attached to the edges of right panel 202 and left panel 204 along each panel's length, configured to bring the edges of the tissue opening toward each other. In one embodiment, a continuous strap comprises a first end 214 and a second end 216. The strap may be placed into tension by pulling first end 214 and second end 216, such that the tensioned strap exerts a laterally compressive force on right panel 202 and left panel 204 and thereby the tissue panels they are applied on. The laterally compressive force may promote healing while inhibiting scar formation. In one embodiment, first end 214 and second end 216 may be secured by any means known to one skilled in the art including but not limited to ties. In one embodiment, straps may include but are not limited to nylon or polypropylene line, suture material, string, a cable, a wire, or other similar items.

In one embodiment, closure member 206 may comprise a series of lateral ties attached to the edges of right panel 202 and left panel 204, configured to bring the edges of the tissue opening toward each other. In one embodiment, closure member 206 may comprise a plurality of independent lateral ties fixed to one panel and being adjustably attachable to the other panel. In one exemplary embodiments, the adjustably attachable end may comprise a ratchet tightening mechanism or similar structure which allows each lateral tie to be independently adjusted at a different spacing between right and left panels 202 and 204. In this way, right and left panels 202 and 204 may be differentially tensioned along their inner edges in order to control and optimize the forces applied to the adjacent tissue edges which are being drawn together. In one embodiment, lateral ties may include but are not limited to nylon or polypropylene line, suture material, string, a cable, a wire, or other similar items.

Biotape 200 may be molded, stamped, machined, woven, bent, welded or otherwise fabricated to create the desired features and functional properties.

### Therapeutic Agents

A therapeutic agent can be in a gas form, a liquid form, a solid form or combinations thereof. In some embodiments, the volume of a therapeutic agent may be in a range of about 0.1 mL to about 50 mL. In certain embodiments, a therapeutic agent of the disclosed bio-zipper 100 is carried in or transported through microstructures 104. An exemplary volume of a therapeutic agent carried within microstructures 104 can be within a range of about 1 nL to about 1 μL.

In accordance with the present disclosure, a therapeutic agent can include one or more agents for delivery after administration/implantation. A wide range of agents may be used. Agents may include, but are not limited to, therapeutic agents and/or an imaging agent. For example, agents may comprise any therapeutic agents (e.g. antibiotics, NSAIDs, angiogenesis inhibitors, neuroprotective agents, chemotherapeutic agents), cytotoxic agents, diagnostic agents (e.g. sensing agents, contrast agents; radionuclides; and fluorescent, luminescent, and magnetic moieties), prophylactic agents (e.g. vaccines), and/or nutraceutical agents (e.g. vitamins, minerals, etc.), or other substances that may be suitable for introduction to biological tissues, including pharmaceutical excipients and substances for cosmetics, and the like. In some embodiments, a therapeutic agent includes one or more bioactive agents.

An agent may comprise small molecules, large (i.e., macro-) molecules, any combinations thereof. Additionally or alternatively, an agent can be a formulation including various forms, such as liquids, liquid solutions, gels, hydrogels, solid particles (e.g., microparticles, nanoparticles), or combinations thereof.

In representative non-limiting embodiments, an agent can be selected from among amino acids, vaccines, antiviral agents, nucleic acids (e.g., siRNA, RNAi, and microRNA agents), gene delivery vectors, interleukin inhibitors, immunomodulators, neurotropic factors, neuroprotective agents, antineoplastic agents, chemotherapeutic agents, polysaccharides, anti-coagulants, antibiotics, analgesic agents, anesthetics, antihistamines, anti-inflammatory agents, vitamins and/or any combination thereof. In some embodiments, an agent may be selected from suitable proteins, peptides and fragments thereof, which can be naturally occurring, synthesized or recombinantly produced.

In some embodiments, an agent can comprise a cell. Such a device can be useful for the injection of whole cells (e.g., stem cells). In some embodiments, an agent comprises a biologic. Examples of biologics including, but are not limited to, monoclonal antibodies, single chain antibodies, aptamers, enzymes, growth factors, hormones, fusion proteins, cytokines, therapeutic enzymes, recombinant vaccines, blood factors, and anticoagulants. Exemplary biologics suitable for use in accordance with the present disclosure are discussed in S. Aggarwal, Nature Biotechnology, 28:11, 2010, the contents of which are incorporated by reference herein.

A therapeutic agent used in accordance with the present application can comprise an agent useful in combating inflammation and/or infection. A therapeutic agent may be an antibiotic. Exemplary antibiotics include, but are not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof. Other anti-microbial agents such as copper may also be used in accordance with the present invention. For example, anti-viral agents, anti-protazoal agents, anti-parasitic agents, etc. may be of use. Additionally or alternatively, a therapeutic agent may be an anti-inflammatory agent.

A therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. In another example, an antibiotic may be combined with an inhibitor of the enzyme commonly produced by bacteria to inactivate the antibiotic (e.g., penicillin and clavulanic acid).

In some embodiments, a therapeutic agent may be any therapeutic gene as known in the art. In some embodiments, a therapeutic agent is a non-viral vector. Typical non-viral gene delivery vectors comprise DNA (e.g., plasmid DNA produced in bacteria) or RNA. In certain embodiments, non-viral vectors are used in accordance with the present invention with the aid of a delivery vehicle. Delivery vehicles may be based around lipids (e.g., liposomes) which fuse with cell membranes releasing a nucleic acid into the cytoplasm of the cell. Additionally or alternatively, peptides or polymers may be used to form complexes (e.g., in form of particles) with a nucleic acid which may condense as well as protect the therapeutic activity as it attempts to reach a target destination.

Alternatively, a therapeutic agent can include one or more surfactants. Various surfactants are known in the art and can be suitable for use as an enhancer to increase tissue permeability for delivery.

A therapeutic agent used in accordance with the present application can comprise an agent useful in promoting cell migration and proliferation.

### Coatings

In accordance with the present disclosure, bio-zipper 100 and biotape 200 can comprise a coating. In some embodiments, the surface of bio-zipper 100 and biotape 200 is coated. In some embodiments, a portion of bio-zipper 100 is coated, such as one or more microstructures 104. In some embodiment, a portion of biotape 200, such as at least one of right panel 202 and left panel 204 is coated. In some embodiments, base 102 is coated. It will be appreciated that a coating may comprise one or more materials/units/layers.

In some embodiments, a coating comprises a payload, which may include one or more agents for delivery. A coating may be a medicated coating being made of or including an agent such as an anti-microbial agent. For example, an anti-microbial agent (e.g., gentamicin, clindamycin, copper, copper ions, silver) and/or a material with an ability to induce anti-microbial activity (e.g., gold that can be heated with an electromagnetic, magnetic, or electric signal) can be coated onto a device or a portion of a device. In another example, a coating can be utilized to carry a payload/agent. In certain embodiments, an agent can be associated with individual layers of a multilayer coating for incorporation, affording an opportunity for exquisite control of loading and release from the coating. For instance, an agent can be incorporated into a multilayer coating by serving as a layer.

In some embodiments, a coating comprises a targeting material such as antibodies, aptamers). Such coatings or materials can be used in combination with any other coating disclosed therein.

In some embodiments, a coating comprises an adhesive material as discussed above. For example, a coating can comprise a bioadhesive such as chitosan and carbopol. Such coatings or materials can be used in combination with any other coating disclosed therein.

Method of Use

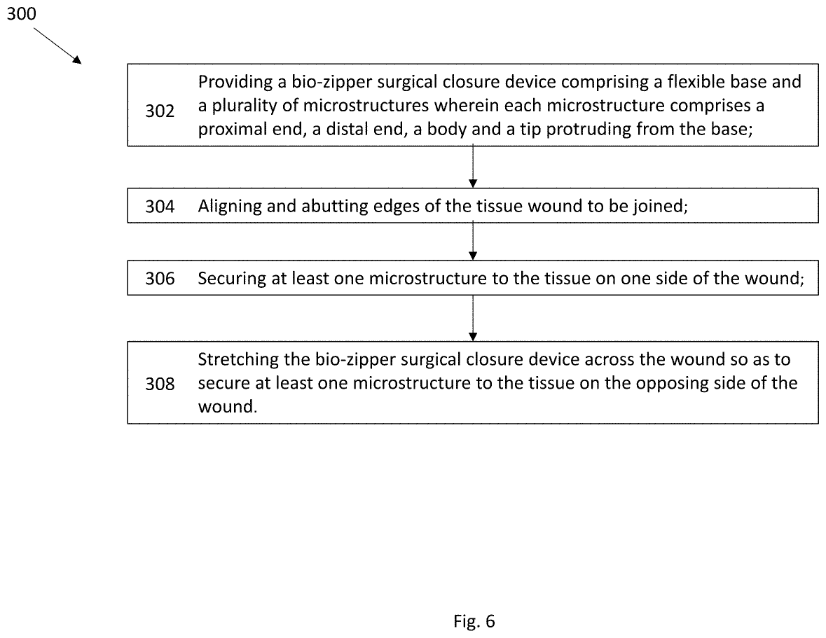

The present invention also relates to methods for the closure of various wounds. Referring now to FIG. 6, an exemplary method 300 of wound closure using the bio-zipper surgical closure device 100 is depicted. Method 300 begins with step 302, wherein a bio-zipper surgical closure device is provided, the bio-zipper surgical closure device comprising a flexible base and a plurality of microstructures, each microstructure comprising a proximal end, a distal end, a body and a tip protruding from the base. In step 304, abutting edges of a tissue wound to be joined are aligned adjacent to each other. In step 306, at least one microstructure is secured to the tissue on one side of the wound.

In step 308, the bio-zipper surgical closure device is stretched across the wound so as to secure at least one microstructure to the tissue on the opposing side of the wound.

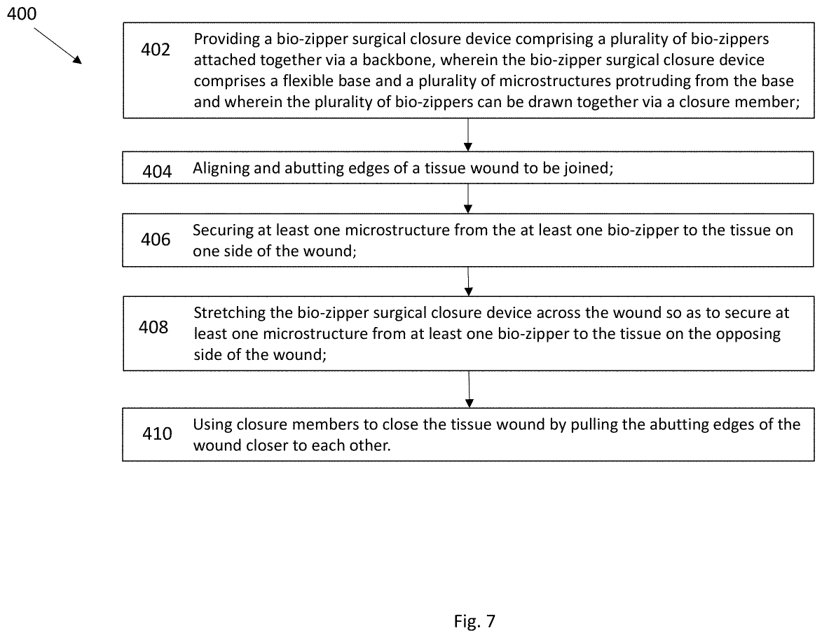

Referring now to FIG. 7, another exemplary method 400 of wound closure using the bio-zipper surgical closure device 100 is depicted. Method 400 begins with step 402, wherein a plurality of bio-zipper surgical closure devices attached together via a backbone is provided, the bio-zipper surgical closure device comprising a flexible base and a plurality of microstructures protruding from the base. In step 404, abutting edges of a tissue wound to be joined are aligned adjacent to each other. In step 406, at least one microstructure from the at least one bio-zipper is secured to the tissue on one side of the wound. In step 408, the bio-zipper surgical closure device is stretched across the wound so as to secure at least one microstructure from the at least one bio-zipper to the tissue on the opposing side of the wound. In step 410, closure members are used to close the tissue wound by pulling the abutting edges of the wound closer to each other.

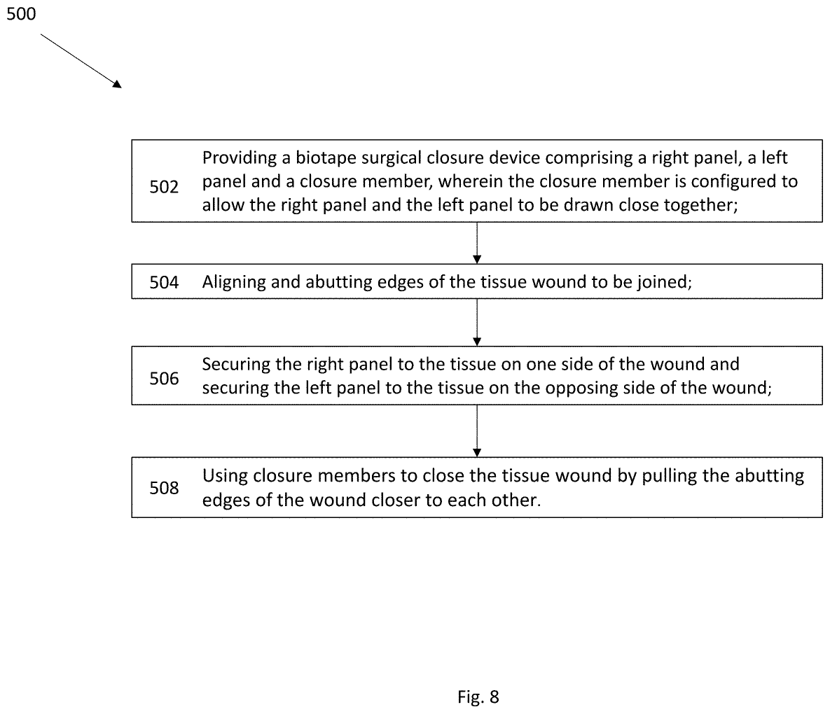

Referring now to FIG. 8, another exemplary method 500 of wound closure using the biotape surgical closure device 200 is depicted. Method 500 begins with step 502, wherein a biotape surgical closure device comprising a right panel, a left panel and a closure member is provided. In step 504, abutting edges of a tissue wound to be joined are aligned adjacent to each other. In step 506, the right panel is secured to the tissue on one side of the wound and the left panel is secured to the tissue on the opposing side of the wound. In step 508, closure members are used to close the tissue wound by pulling the abutting edges of the wound closer to each other.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### Example 1

### Bio-Zipper Surgical Closure Device

Congenital and acquired conditions including neurogenic bladder, bladder exstrophy, and bladder cancer can result in the need for surgical lower urinary tract reconstruction (LUTR) to maintain a functional urine reservoir (ACS 2020. Key statistics for bladder cancer Atlanta, GA: ACS; Horst M. et al., 2019, Front Pediatr., 7(91):1-12). These complex surgical procedures typically utilize a segment of bowel to replace the bladder (e.g., neobladder, conduit; U.S. 20,000/year) (ACS 2020. Key statistics for bladder cancer Atlanta, GA: ACS) or expand its capacity (e.g., augmentation cystoplasty; U.S. 1,400/year) (Lendvay, T.S. et al., 2006, J. Urol., 176(4):1716-1720; Szymasnki, K.M. et al., 2019, J. Urol., 202(6):1256-1262; NINDS. Spina Bifida Fact Sheet Bethesda, MD: BRAIN; 2020). Early postoperative complications following the current sutured anastomoses are common (30%-60% in 30 days), the incidence of which may be altered by tissue characteristics and surgical factors (Sturm, R. M. et al., 2016, Curr. Bladder Dysf. Rep., 11:225-233; Du, K. et al., 2012, J. Ped. Surg., 50:1535-1539; McNamara, E. R. et al., 2015, J. Ped. Urol., 11(209):e1-6).

Recent developments in LUTR include an increased appreciation for the role of surgeon experience (Barbieri, C.E. et al., 2007, J. Urol., 178:1418-1422) and the value of robotic-assisted procedures with intracorporeal anastomoses (procedure completed entirely within the abdominal cavity through several small incisions) to decrease procedural risk and patient morbidity. In the study, LUTR was selected as an optimal clinical context for testing and validation of a repair device based on extensive customer segment interviews with surgeons, including pediatric urologists and urologic oncologists across all levels of experience and practice settings, with expertise in various intra-abdominal and pelvic reconstructive procedures (general, colorectal, minimally invasive, urologic and gynecologic surgery). While robotic intracorporeal LUTR decreases blood loss, decreases perioperative patient morbidity (e.g. bowel and wound complications, length of hospital admission), and has non-inferior major complication rates, mortality and oncologic outcomes when compared to open procedures (Ahmed, K. et al., 2014, J. Urol., 65:e918; Collins, J. W. et al., 2017, Eur. Urol., 71:723-726), its utilization remains limited for LUTR (<40%) largely due to challenging and time-consuming sutured anastomoses. Further, when a robotic approach is selected by surgeons with low cystectomy case volume, the patient more commonly proceeds to an incontinent diversion as compared to high-volume centers in which continent diversions may be more commonly performed (Hautmann, R. E. et al., 2015, Urology, 85:233-238). The surgical closure device addresses this identified practice gap directly via the development of a biomimetic patch with mechanical properties of its backbone tuned to those of the tissue to which it has been applied. Its unique backbone structure, adhesive layer, and design not only provide equal distribution of tension across the repair but also acts as a sealant and support mechanism. Thus, the device has the potential to improve quality of life and outcomes for children and adults undergoing LUTR via rapid, consistent closures that will facilitate the transition to minimally invasive, intracorporeal, patient-driven diversion selection.

Clinical Problem

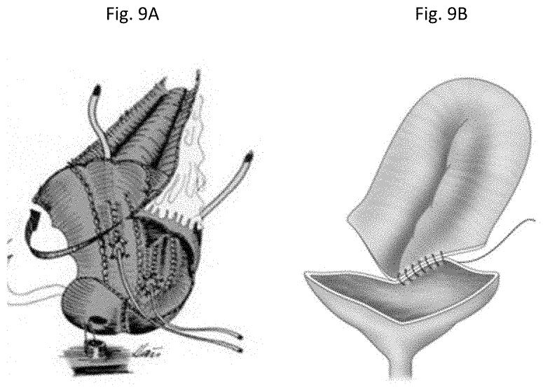

Lower urinary tract reconstruction (LUTR) represents a range of procedures (FIG. 9A and FIG. 9B) in which the quality of the surgical closure is a major determinant of clinical outcomes and is an opportunity to expand complex robotic surgery in a patient and surgeon cohort optimally positioned to achieve maximum value in the transition to a robotic-assisted laparoscopic approach.

Lumens throughout the body frequently require repair, representing procedures as diverse as vascular anastomoses in a transplant to management of a congenital esophageal condition. The current gold standard for these repairs is often a sutured surgical closure in multiple layers to decrease risk of complications such as leak, fistula, or erosion. The lack of surgical repair options is particularly evident in the urinary tract where permanent staples or stents may serve as a nidus for stone formation or infection, thus limiting the use of rapidly applied, consistent closure methods that have become standard of care in other fields such as gastrointestinal surgery. The tedious nature of sutured urinary tract reconstructive procedures limits a surgeon's ability to rapidly apply their open surgical skills in the adoption of novel approaches, a finding that is increasingly relevant as the repertoire of minimally invasive techniques required by new robotics platforms will continue to expand. The surgical closure device of the present invention is specifically developed to decrease time and improve the consistency and quality of internal luminal repairs. With embodiments for both open and minimally invasive (laparoscopic, robotic-assisted) deployment, this device plays a vital role in facilitating the current trend toward minimally invasive surgical procedures by decreasing the learning curve to acquire the unique skills required for complex suture line completion in these settings.

Addresses Complex Procedures in a High-Risk Cohort for Perioperative Morbidity

Cystectomy and LUTR are high-risk procedures performed in high-risk patients and are the costliest procedures performed in urology. The most common condition leading to LUTR is bladder cancer, the sixth leading cause of cancer in the United States (80,000 diagnosed/year; 20,000 cystectomies/LUTR/year) (team TACSmaec, Key statistics for bladder cancer Atlanta, Ga.: ACS; 2020). The typical patient undergoing these procedures for bladder cancer is elderly (mean age 73 years at diagnosis), often recently completed neoadjuvant chemotherapy and has multiple comorbidities (most common risk factor: smoking) (team TACSmaec, Key statistics for bladder cancer Atlanta, Ga.: ACS; 2020), all of which increase perioperative morbidity. Children undergoing LUTR for neurogenic bladder are often wheelchair bound due to spinal cord conditions and have truncal obesity and mobility limitations that likewise increase their perioperative risk (Donovan, B. O. et al., 2009, The Journal of Urology 181(5):272-2276). Overall, LUTR has a 30%-60% 30-day reported complication rate, with the majority attributable to surgical factors (Sturm, R.M. et al., 2016, Curr. Bladder Dysf. Rep., 11:225-233; Du, K.M. et al., 2012, J. Ped. Surg., 50:1535-1539; McNamara, E.K. et al., 2015, J. Ped. Urol., 11(209):e1-6; Barbieri, C.L. et al., 2007, J. Urol., 178:1418-1422). A device that can improve the consistency of surgical repairs, thus minimizing risk to this high-risk cohort would be poised to have significant impact in this space.

Facilitates ongoing Urologic Surgery Transition to Robotic-Assisted Procedures while Decreasing the Effect of Low Volume on Surgical Outcomes

Between 2009 to 2015, the robotic-assisted proportion of radical prostatectomies increased from 58.9% to 80.8% (p<0.001); cystectomies increased from 11.6% to 25.0% (p<0.001) (Mazzone, E. et al., 2019, Lap. Robotic Surg., 33(6):438-447). These same surgeons who have rapidly transitioned to replace the open prostatectomy with robotic-assisted techniques are the target users of the Bio-Zipper, a cohort with extensive training in robotics and demonstrated availability of the technology within their institutions. However, it is important to note that although these surgeons are skilled in robotic surgery, their cystectomy experience is often limited. American Board of Urology case logs demonstrate that 54% of surgeons who performed cystectomies completed only 1 in a 6-month period (Flum, A. S. et al., 2015, Uro. Practice, 2:367-372). Only 10% performed ≥5, consistent with the minimum case volume to achieve the threshold effect of lowest mortality rate in line with consensus recommendations (McCabe, J. E. et al., 2007, Postgrad. Med., 83:556-560). In fact, nationwide the majority of robotic radical cystectomies by volume are performed in low-volume, non-academic centers, a factor associated with longer initial hospitalizations, increased re-hospitalizations, and elevated perioperative mortality (Barbieri, C. E. et al., 2007, J. Urol., 178:1418-1422). Thus, to ensure optimal outcomes, straightforward solutions that decrease the learning curve and skill maintenance to complete complex portions of such procedures are needed. Surgeons who are trained in robotics in a field that has already taken advantage of its clinical benefits are ideally suited to utilize a device that meets reconstructive needs across a variety of skill levels and robotic platforms.

Expands Demonstrated Clinical Benefit of Robotic Assisted Intracorporeal LUTR

There are two general approaches to bowel management to prepare the tissue for placement in the urinary tract during a robotic procedure. The bowel can either be reconfigured with extensive suture line completion by delivering it outside the body for open suturing (extracorporeal urinary diversion, ECUD), or it can be completed entirely within the abdominal cavity (intracorporeal urinary diversion, ICUD). As this reconfiguration requires approximately 50% or more of the total operative time to complete an LUTR, this aspect is a key factor in the postoperative benefit that the patient receives from the procedure. Although ECUD decreases perioperative blood loss (p<0.001) and wound complications (p=0.03), an ICUD decreases these further while also minimizing postoperative time to return of bowel function and gastrointestinal complications (10% ICUD vs 23% ECUD, p<0.001), key components for early recovery (Ahmed, K. K. et al., 2014, Eur. Urol., 65:e918; Collins, J. W. et al., 2017, Eur. Urol., 71:723-726). Further, ICUD is the only approach in which patients with cardiopulmonary disease did not have a demonstrated increase in hospital stay or major complications compared to patients without these comorbidities (Lamb, B. W. et al., 2016, Urol. Oncol., 34(417):e17-23). Despite this advantage, ICUD requires additional OR time in comparison to ECUD (mean total time 567 vs 510 minutes, p=0.002). A device that decreases the time for bowel reconfiguration and suturing would thus address a major barrier to ICUD adoption and facilitate its evidence-based adoption.

Supports Patient-Physician shared Decision making in Diversion Selection

A consensus recommendation is that patients undergoing LUTR receive counseling regarding diversion options, which often includes an incontinent diversion to a stoma covered with a bag on the abdominal wall (e.g., ileal conduit) versus a continent diversion with rerouting through the urethra to facilitate future voiding through the natural orifice (e.g., neobladder). However, the data indicates that the major factor associated with diversion selection is in fact the surgeon and hospital where these complex reconstructions occur. Therefore, even though low-volume U.S. surgeons are most commonly performing robotic cystectomies in combination with incontinent diversions, patients rarely receive neobladders (<15%) in this setting, compared to up to 75% of those receiving care in centers most familiar with this surgical option (Hautmann, R. E. et al., 2015, Urology, 85:233-238). Due to patient factors, a continent diversion may not be an option for all patients; however, when it is a device that removes barriers to continent diversions can expand treatment options in line with patient preferences.

Substantially decreases Total Cost of Robotic LUTR

Based on the data regarding improvement in outcomes balanced against a projected device cost of $1,000, and surgical time/cost estimates based on an estimated total time savings of 38% per case, robotic neobladder diversion using a Bio-Zipper is estimated to decrease the cost of robotic LUTR by $10,983/case in adults ($54,128 to $43,145) and $16,903/case in children ($59,184 to $42,281) undergoing an augmentation cystoplasty in the 30-day perioperative period. Assuming an estimated increase to 60% robotic utilization that will occur regardless of device utilization, this translates to an annual U.S. total cost savings for robotic LUTR of $143 million.

Standard-of-Care Limitations and Design Challenge

The overarching goal of the Bio-Zipper is to meet the design needs for LUTR to facilitate a fast and consistent application that decreases the time and specialized skill required and integrates within the current workflow of the procedure. Devising a method to perform LUTRs that have adequate tissue support and a watertight closure poses a considerable design challenge. The closure device must seal and support the incision to prevent urine leakage (epithelial inversion), maintain adequate elasticity and minimize hysteresis despite the potential for intermittent distension (Abbas, T. O. et al., 2018, Frontiers in Pediatrics, 5:283). To enable implantation, the material selected must be biocompatible and facilitate normal wound healing. Furthermore, the material must be biodegradable without toxic degradation while remaining in a secure position throughout wound healing. There is currently no device that meets these requirements for internal luminal repair in the urinary tract. The current standard-of-care is a sutured, multilayered closure, requiring significant handling of delicate tissues with the potential for localized wound tension or de-vascularization. Stapled closures are not currently standard-of-care in the urinary tract due to the risk of stone formation or infection associated with permanent titanium staples within the urinary tract. Similarly, chemical adhesives such as cyanoacrylate may release toxic degradation products. At present, fibrin glue and biocompatible hydrogels have demonstrably lacked adhesive strength and flexibility to remain watertight despite intermittent distension. An option is needed to address each aspect of this design challenge for optimal luminal closure while simultaneously decreasing surgical variability and improving outcomes following LUTR.

### Innovation: Technology Description and Novelty

The device of present invention is designed to facilitate epithelial inversion, minimize urine leak, and alleviate localized tension along suture lines while providing mechanical strength and elasticity that mimics that of underlying bowel and bladder tissue to allow intermittent luminal distension. As indicated in FIG. 3, FIG. 4 and FIG. 5, the device is composed of (1) a flexible backbone with tunable mechanical properties, (2) a bio-adhesive layer, and (3) a mechanism for pulling the segments of the device together.

Whether an open or minimally invasive procedure is performed, the Bio-Zipper is positioned at the time of tissue edge to edge approximation. This device may be placed following an initial intermittent (interrupted) suture placement for tissue approximation with standard retraction to align the tissue. Once affixed to the tissue on each side of the defect, the two aspects will be “zipped” together via the central portion, which will be optimized for rapid closure using a single suture drawstring or snap mechanism which will facilitate epithelial inversion during this closure process. To account for potential length discrepancy of the tissue being approximated, the device can be placed in tandem using variable device section lengths with or without locking devices together in the longitudinal direction.

The unique structure and design of the device not only provides equal distribution of tension across the wound, but also serve as a sealant and support mechanism. The addition of an adhesive mechanism to this backbone will prevent migration and improve stability throughout the healing process. The backbone may additionally be modified in midline as needed with a mesh-like structure to allow overlying placement of omental coverage and is suturable (through the material versus with eyelets) for secondary fixation of the device as desired. Finally, the device minimizes any local effects of tissue necrosis and de-vascularization by avoiding the need for a running, multilayered suture line.

The materials and methods employed in these experiments are now described.

Material Selection, device Backbone

The backbone of the device needs to be flexible and elastic in line with human bowel and bladder mechanical properties. It also needs to be bioresorbable to facilitate wound healing without a chronic inflammatory or fibrotic local tissue response.

Synthesis

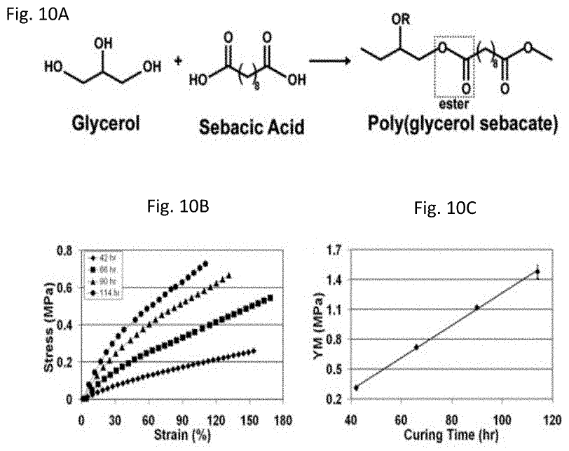

To address these needs, Poly(glycerol sebacate) (PGS) is used as the primary component of the device backbone. PGS is a well-established biomaterial that was designed to mimic the mechanical behavior of extracellular matrix components collagen and elastin (Pomerantseva, I. et al., 2009, J. Biomed Matl. Res. Part A, 91A(4):1038-1047; Shul, D. J. et al., 2018, Regenerez Elastomeric Properties White Paper. Telford, PA: The Secant Group). PGS is synthesized by the polycondensation of glycerol and sebacic acid (FIG. 10A). This creates a viscous prepolymer that can be further crosslinked through increasing the temperature past the prepolymer melting point to covalently crosslink and stabilize the polymer structure (Pomerantseva, I. et al., 2009, J. Biomed Matl. Res. Part A, 91A(4):1038-1047).

Mechanical Properties

The PGS synthesis process can be leveraged to tune the mechanical properties of the final elastomer. The mechanical properties of PGS is tuned by altering the ratio of glycerol and sebacic acid during the initial synthesis step, by altering the temperature in the secondary curing step, or by modifying crosslinking time in the secondary curing step (Smoot, C. J. et al., 2018, Regenerez Degradation and Release Kinetics White Paper. Telford Pa.: The Secant Group). Increasing the crosslinking temperature or curing time increases the crosslinking density, thus increasing the stiffness of the PGS elastomer (FIG. 10B and FIG. 10C) (Pomerantseva, I. et al., 2009, J. Biomed Matl. Res. Part A, 91A(4):1038-1047; Smoot, C. et al., 2018, Regenerez Degradation and Release Kinetics White Paper. Telford Pa.: The Secant Group). Additionally, previous studies have demonstrated PGS implantation in both subcutaneous and intramuscular models resulting in minimal fibrotic and inflammatory response, regardless of glycerol: sebacic acid ratio or crosslinking parameters (Pomerantseva, I. et al., 2009, J. Biomed Matl. Res. Part A, 91A(4):1038-1047).

Adhesive Selection

To minimize risk of delamination and to improve mechanical stability of the sealant material, the focus of development is tissue adhesion with mechanical stability imparted by the synergy between adhesion and cohesion with optimization for hydrated tissue despite the presence of localized blood during the procedure. It is recognized that both mechanical and chemical environments drive the adhesive properties of hydrogels.

From a mechanical viewpoint, interlocking between sealant hydrogels and uneven surface morphologies of tissues favors the adhesion of the fit-to-shape sealants through the chemical crosslinking of hydrogel precursors. On the other hand, hydrophilic functional groups present on the surface of proteins may contribute to improved integration of the material with the underlying tissue substrate during the polymerization process (Ghobril, C. et al., 2015, Chem. Soc. Rev., 44(7):1820-1835; Yang, J. et al., 2020, Advanced Functional Materials, 30(2):1901693).

To engineer an adhesive patch with an elastic backbone and adhesive surface, both cohesion with the tissue and chemical bonding of the surfaces by functionalizing the backbone material is exploited. The groups are then covalently bond to the abundant amine groups on the tissue.

The following strategies are evaluated to form an adherent modified-PGS substrate: A) Incorporating hydrophilic groups on the backbone of the PGS will make the material compatible to enable covalent binding of the desired peptides via amide bonding. To enable carboxyl group incorporation, a strategy to modify the polymer has been successfully developed involving sequential treatment of alkaline hydrolysis followed by acidification. In this manner, the amine groups on the tissue surface and carboxylate groups on PGS can interact with each other in the presence of NHS/EDC coupling agents (COOH-PGS is decorated with NHS and applied to the tissue for adhesion). B) The hydroxyl groups of PGS are converted to amine groups using (3-Aminopropyl) triethoxysilane. Then, another crosslinking molecule with NHS on both ends (e.g. Bis(NHS)PEG5) is used to adhere to the amine groups on the tissue.

Other modifications are used for strong adhesion during in vitro evaluation. The following strategies provide additional hydroxyl groups on the backbone of sebacic acid, thus creating a new material with desired adhesion properties. I) Replace glycerol with tannic acid. This enables two mechanisms of action, 1) catechol adhesion directly to tissue, 2) increased hydroxyl group presence on the backbone provides more functional groups for the crosslinkers to have stronger adhesion of membrane with tissue. II) Replace glycerol with branched PEG. This enables increased hydroxyl group density. This material is called PEGylated Poly(glycerol sebacate) (Wang, Y. et al., 2019, Polymers, 11(6)).

Animal Model Tissue Selection

A rat model is selected for biocompatibility evaluation of the patch-adhesive composite in line with historic utilization for subcutaneous testing (Pomerantseva, I. et al., 2009, J. Biomed Matl. Res. Part A, 91A(4):1038-1047; Annabi,

N. et al., 2017, Biomaterials, 139:229-243). Porcine tissue evaluation is used due to its use as the proposed animal candidate model for future in vivo studies due to suitability for minimally invasive LUTR and tissue mechanical properties. The porcine bladder has similar ultimate tensile strength (UTS) and elastic modulus (EM) to human bladder tissue (Porcine bladder UTS 0.32±0.53kPa, Human SSM 0.64±0.34kPa, p=0.6;(Stewart, D. B.

et al., 2018, PLOS One, 13(7)) Colon: Porcine SSM 0.56±0.24, Human SSM 0.70±0.46 kPa, p=0.7) (Stewart, D. C. et al., 2018, PLOS One, 13(7)). Due to their mechanical properties, model suitability for robotic surgery utilizing the da Vinci technology and prior demonstration of accurate and realistic simulation of pneumoperitoneum, porcine bladders are used throughout the study for in vitro and ex vivo evaluation (Dawda, S. et al., 2019, J. Med. Syst., 43(10):317-331).

### Methods and Approach

Engineer a patch that mimics mechanical properties of native bladder tissue

bladder and small bowel (ileum) are evaluated to establish tissue mechanical characteristics followed by evaluation of PGS (Table 1) with varied ratios of glycerol and sebacic acid (0.7 to 1.3 G: SA) and crosslinking parameters (42 to 114 hours) (Pomerantseva, I. et al., 2009, J. Biomed Matl. Res. Part A, 91A(4):1038-1047). Following each modification, physical testing and verification of chemical characteristics is completed. Candidate PGS parameters for adherence modifications is selected such that target tensile modulus is within 50kPa to 250kPa of tissue parameters; degradation profile is 75% to 100% mass loss in six weeks.

TABLE 1
Overview of Proposed Material and Device Testing

| Test Type | Study | Aims | Notes |
|---|---|---|---|
| Physical | Gross | 1A, 1B | Material, Device |
| | Description | | |
| | NMR | 1A, 1B | Material, Device |
| | Spectroscopy | | |
| | Light | 1A, 1B | Material, Device |
| | microscopy | | |
| | Scanning | 1A, 1B | Material, Device |
| | Electron | | |
| | Microscopy | | |
| | Swelling | 1B | Device |
| | Degradability | 1B | Device |
| | Suturability | 1B | Backbone |
| Mechanical | Tensile Modulus | 1A, 1B | Backbone, Closure |
| | Tensile Strength | 1A, 1B | Backbone, Closure |
| | Extensibility | 1A, 1B | Backbone |
| | Compression | 1B | Backbone |
| | Adhesion | 1B | Static and Dynamic, Radial |
| | | | and Longitudinal |
| Biologic | Cell viability | 1B | In vitro fibroblast cell |
| | | | culture |
| | Biocompatibility | 1B | Rat Subcutaneous |
| | | | Implantation |
| Surgical | Ex vivo | 2A | Open, Laparoscopic, Robotic |
| | technical | | Deployment |
| | Ex vivo | 2A | Surgeon Learning Curve, |
| | technical | | Time, Accuracy |
| | Ex vivo efficacy | 2A, 2B | Gross and Histologic Closure |
| | | | Evaluation |
| | Ex vivo efficacy | 2B | Functional Closure Evaluation |
| | | | (Urodynamics) |

Characterize the Mechanical, Physical, and Biological Properties of PGS-Adhesive Patch and Patch-Tissue Composites

As noted in ‘adhesive selection’ above, PGS is modified in a stepwise fashion with complete evaluation of physical and mechanical effects of the PGS-adhesive modified material. In addition to the characterization described for PGS, suturability is evaluated using qualitative tear testing. Following selection of a PGS-based adhesive, a manufacturing process demonstrated as feasible for PGS microneedle fabrication is utilized to create the device for tissue closure application, utilizing a laser-etched poly(methyl methacrylate) (PMMA) mold.

Adhesive Characterization

Adhesive characterization is completed using thickness frozen porcine small bowel (ileum) and bladder (2×2cm) samples in semi-dry and wet conditions. Adhesion to tissue is evaluated in a static fashion on a single tissue sample, followed by evaluation across two samples simulating surgical repair. The device closure is compared to sutured repairs, staples (titanium) and cyanoacrylate glue. Using a tensile testing machine (Universal Testing System, Instron), the force required to remove the adhesive from the tissue or to repair failure respectively and the failure mechanism is noted. Finally, dynamic adhesion testing is completed to evaluate adhesion when exposed to shear stress with 100 cycles of applied stress per sample (Yang, S. Y. et al., 2013, Nat. Commun., 4:1-16; Annabi, N. et al., 2017, Sci. Transl. Med., 9:1-14; Luo, Z. et al., 2019, Adv. Health Mater., 8(3):e1801054; Pok, S. et al., 2013, Acta Biomat., 9(3):5630-5642; Zhao, X. et al., 2016, Adv. Health Mater., 5(1):108-118).

Cell Viability

Cell viability is determined in vitro using commercial live/dead kit, Actin/DAPI staining and PrestoBlue assays to evaluate cell viability, spreading and metabolic activity, respectively. Two-dimensional culture of NIH-3T3 mouse embryonic fibroblast cells is evaluated at days 1, 3 and 5 post-seeding (Annabi, N. et al., 2017, Biomaterials, 139:229-243).

Subcutaneous implantation is performed to evaluate the in vivo biocompatibility of the adhesive-patch composite. Three, 2cm-long midline incisions is made on the back of adult male and female Sprague Dawley rats to create bilateral subcutaneous pockets by blunt dissection. Sample groups include 1. Sham 2. Sham with tacking suture in muscle. 3. PGS patch 4. PGS with tacking suture. 5. PGS adhesive 6. PGS adhesive w/ tacking suture. The rats are euthanized with excision of implants and surrounding tissue postoperative day 3, 7, 14, 28 and 56 (Annabi, N. et al., 2017, Biomaterials, 139:229-243). Histologic and immunofluorescent staining is performed with quantification of inflammatory markers (primary antibodies CD68, 206, 86, MPO, IL1β, TNFα, IL-10, IL-13) (Bury, M.I. et al., 2014, Biomaterials, 35(34):9311-9321).

Statistical Analysis

Based on prior analyses of adherence, n=5 samples are evaluated per adhesion and mechanical evaluation; n=6 animal models per endpoint are evaluated (3 female, 3 male; 5 endpoints for a total of 30). Mean and Standard deviation is reported. For multiple comparisons, analysis of variance is performed with the Tukey's honestly significant difference test (significance 95%).

Anticipated Results, Potential Pitfalls and Alternative Approaches

For mechanical testing, a material is selected with parameters that mimic the mechanical properties of porcine bowel and bladder tissue. A potential pitfall is that

PGS mechanical properties is affected by chemical modifications for adhesion. To ensure that parameters remain within the specified range, PGS is evaluated with and without the adhesive component with further refinement as required to achieve the parameters described above. Another potential pitfall is that of adequate adherence to tissue, particularly in the setting of moist tissue and/or repeated distension cycles required in the urinary tract. An alternate method that may be utilized is the application of PGS-co-PLA as glue brushed onto the surface of PGS membrane followed by tissue application (Chen, Q. et al., 2011, Soft Matter., 7(14):6484-6492).

Evaluate the Biomimetic Patch during ex vivo Application to Bladder and Neobladder Pepair Models

Significance

A method for deployment in a minimally invasive environment is optimized during CASIT evaluation. The result is a novel platform for urinary tract application, resulting in rapid deployment, decreasing time and variability in sutured closures.

### Methods and Approach

Optimize Surgical Open and Robotic Patch Application and Laparoscopic Port Deployment

Following practice and optimization of placement in open, laparoscopic and robotic fashion, the PI creates a video-taped training module for each mode of application and the standard-of-care sutured closure. After watching this module, surgeons are observed completing the repairs using each mode of application to tissue. For minimally invasive application, the tissue is secured in a laparoscopic box trainer. Effects of the surgical closure device on time to complete each repair (sutured standard of care versus surgical closure device of this invention), learning curve by experience: 1. Surgical resident trainees, 2. Low-volume robotic (<8 LUTR/year), 3. High-volume robotic (≥8 LUTR/year), 4. Low-volume open (<8 LUTR/year), 5. High-volume open ((≥8 LUTR/year), surgeon description of satisfaction and ease of use (qualitative and quantitative debriefing survey) are evaluated. Task completion order is randomized.

Encounters are videotaped followed by surgeon skill scoring by blinded peers with and without surgical closure device of this invention (Global Evaluative Assessment of Robotics Skill parameters: depth perception, bimanual dexterity, efficiency, force sensitivity, autonomy, robotic control) (Goh, A.C. et al., 2012, J. Urol., 187(1):247-252). Tissue is evaluated following each repair grossly and histologically to evaluate evidence of tissue injury and approximation efficacy including effective epithelial inversion.

Complete Urodynamic Evaluation of Luminal Distension and Closure Integrity in ex vivo and in vivo Tissue.

Explanted porcine small bowel and bladders from healthy animals are incised, reconfigured, and repaired in 1) a standard of care running and imbricated layered sutured closure or 2) closure utilizing the surgical closure device of present invention. A urinary catheter is placed into the bladder via the urethra with watertight tie around the catheter and of the ureters to obtain intravesical measurements. The cannula exiting the catheter hub is connected to a syringe pump (e.g., Elite Syringe Pumpll, Harvard Apparatus) and to a physiological pressure transducer and bridge amplifier. The bladder is filled with 37° C. PBS with methylene blue at a rate of 5 to 10% capacity with continuous monitoring of intravesical pressure (Bury, M.I. et al., 2014, Biomaterials, 35(34):9311-9321). Volume infused and pressure at time of device or suture repair failure is recorded; a cohort of bladders will additionally be utilized for 50 repeat cycles of filling to 50 and 100% capacity to determine efficacy of device with physiologic filling conditions.

### Statistical Analysis

A minimum of 3 tissue anastomoses per cohort and closure method is evaluated, consistent with a primary aim of determining intravesical pressure at time of closure failure (3 bowel to bladder, 3 bladder to bladder, 3 bowel to bowel) (Bury, M. I. et al., 2014, Biomaterials, 35(34):9311-9321). Each surgeon completes a minimum of five repetitions to evaluate a validated calculated score across occurrences (Olthof, E. et al., 2008, The Learning Curve of Robot-Assisted Laparoscopic Surgery, Medical Robotics. Bozovic V, editor. Croatia: InTech).

### Results, Potential Pitfalls and Alternative Approaches

Standard surgical techniques may require modification to allow adequate tissue retraction and tension for device application. The passage of the device through the trocar is not anticipated to be of significant concern as it is flexible and small enough to allow passage through a cannula to avoid damage within the trocar. The early surgeon feedback and video observations is the key to refine the device for future applications; it is possible to add a secondary layer that is removed if necessary, once the device is positioned on the tissue to avoid these concerns. Additional fixation and potential for suture tacking if desired by surgeons needs to be tested in this environment. If required, patterned curing can be utilized to reinforce regions that may require increased handling or suturing to avoid damage during placement. Both device and surgeon handling is evaluated during the procedures to inform the need for regions of increased reinforcement. Finally, if there are challenges creating a watertight closure within the environment, varying shapes can be examined for external support and ensure epithelial inversion, hydrophobic regions to minimize urine leak at the edge of the device and/or adjust the location or application of adhesive to ‘seal’ the midline incision line prior to device closure.

The surgical closure device of the present invention is evaluated for additional luminal procedures such as esophageal or bowel anastomoses, vaginal cuff, cardiac or vascular repairs. The application may include the closure of soft, planar, inner organs (e.g. liver, pancreas, kidney) and that the device can facilitate minimally invasive procedures in a manner that would minimize or eliminate the need for robotic assistance. Finally, device of the present invention can be used in applications for localized therapeutic delivery and monitoring of a surgical site by taking advantage of the unique properties of this implantable surgical device with tunable degradability.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention.

The appended claims are intended to be construed to include all such embodiments and equivalent variations.

What is claimed is:

1. A bio-zipper surgical closure device comprising:
a flexible base; and
a plurality of microstructures, each comprising a proximal end, a distal end, a body and a tip protruding from the base.

2. The bio-zipper surgical closure device of claim 1, wherein the microstructures are selected from the group consisting of microneedles, microblades, microanchors, microfishscale, micropillars, microhairs and combinations thereof.

3. The bio-zipper surgical closure device of claim 1, wherein the microstructures each comprise a tip diameter ranging from about 10 nm to about 1 μm.

4. The bio-zipper surgical closure device of claim 1, wherein the microstructures each comprise a length ranging from about 1 μm to about 2 mm.

5. The bio-zipper surgical closure device of claim 1, wherein the base is biodegradable.

6. The bio-zipper surgical closure device of claim 1, wherein the plurality of microstructures are biodegradable.

7. The bio-zipper surgical closure device of claim 1, wherein the plurality of bio-zipper devices are linked together via a flexible backbone.

8. The bio-zipper surgical closure device of claim 7, wherein the plurality of bio-zipper devices are placed adjacent together leaving a space between each bio-zipper ranging between about 0 to about 1cm.

9. The bio-zipper surgical closure device of claim 1, wherein the plurality of bio-zipper devices are linked together via a closure member, wherein the closure member allows the at least two adjacent bio-zippers to be drawn closer together.

10. The bio-zipper surgical closure device of claim 9, wherein the closure member is selected from the group consisting of a suture, a pull tab and combinations thereof.

11. A biotape surgical closure device comprising:
a right panel;
a left panel; and
a closure member, wherein the closure member is configured to allow the right panel and the left panel to be drawn close together.

12. The biotape surgical closure device of claim 11, wherein the closure member is selected from the group consisting of a suture, a pull tab and combinations thereof.

13. The biotape surgical closure device of claim 11, wherein the right panel and the left panel are made from adhesive material.

14. The biotape surgical closure device of claim 13, wherein the right panel and the left panel comprise Poly (glycerol sebacate) (PGS).

15. A method for wound closure comprising:
providing a bio-zipper surgical closure device, wherein the bio-zipper surgical closure device comprises a flexible base and a plurality of microstructures, wherein each microstructure comprises a proximal end, a distal end, a body and a tip protruding from the base;
aligning and abutting edges of a tissue wound to be joined;
securing at least one microstructure to the tissue on one side of the wound;
stretching the bio-zipper surgical closure device across the wound so as to secure at least one microstructure to the tissue on the opposing side of the wound.

16. The method of claim 15, wherein the microstructures are selected from the group consisting of microneedles, microblades, microanchors, microfishscale, micropillars, microhairs and combinations thereof.

17. The method of claim 15, wherein the microstructures each comprise a tip diameter ranging from about 10 nm to about lum.

18. The method of claim 15, wherein the microstructures each comprise a length ranging from about lum to about 2 mm.

19. The method of claim 15, wherein the base is biodegradable.

20. The method of claim 15, wherein the plurality of microstructures are biodegradable.

21. A method for wound closure comprising:
providing a bio-zipper surgical closure device comprising a plurality of bio-zippers attached together via a backbone, wherein the bio-zipper device comprises a flexible base and a plurality of microstructures protruding from the base and wherein the plurality of bio-zippers can be drawn together via a closure member;
aligning and abutting edges of a tissue wound to be joined;
securing at least one microstructure from the at least one bio-zipper to the tissue on one side of the wound;
stretching the bio-zipper surgical closure device across the wound so as to secure at least one microstructure from at least one bio-zipper to the tissue on the opposing side of the wound;
using closure members to close the tissue wound by pulling the abutting edges of the wound closer to each other.

22. The method of claim 21, wherein the microstructures are selected from the group consisting of microneedles, microblades, microanchors, microfishscale, micropillars, microhairs and combinations thereof.

23. The method of claim 21, wherein the microstructures each comprise a tip diameter ranging from 10 nm to lum.

24. The method of claim 21, wherein the microstructures each comprise a length ranging from about 1 μm to about 2 mm.

25. The method of claim 21, wherein the base is biodegradable.

26. The method of claim 21, wherein the plurality of microstructures are biodegradable.

27. A method for wound closure comprising:
providing a biotape surgical closure device comprising a right panel, a left panel and a closure member, wherein the closure member is configured to allow the right panel and the left panel to be drawn close together;
aligning and abutting edges of a tissue wound to be joined;
securing the right panel to the tissue on one side of the wound and securing the left panel to the tissue on the opposing side of the wound;
using closure members to close the tissue wound by pulling the abutting edges of the wound closer to each other.
